(19) United States
(12) Patent Application Publication
(10) Pub. No.: US 2026/0041451 A1
(43) Pub. Date: Feb. 12, 2026

(54) Surgical Device for Cutting Tissue

(71) Applicant: The Government of the United States, as Represented by the Secretary of the Army, Ft. Detrick, MD (US)

(72) Inventor: Alexander J. Villahermosa, San Antonio, TX (US)

(73) Assignee: The Government of the United States, as Represented by the Secretary of the Army, Ft. Detrick, MD (US)

(21) Appl. No.: 19/100,272

(22) PCT Filed: Sep. 27, 2023
(86) PCT No.: PCT/US2023/033782

§ 371 Date: Jan. 31, 2025

Related U.S. Application Data
(60) Provisional application No. 63/377,613, filed on Sep. 29, 2022.

(51) Int. Cl. A61B 17/3205; A61B 17/02; A61B 17/32; A61B 18/00; A61B 18/14

(52) U.S. Cl. CPC A61B 17/3205; A61B 17/0218; A61B 18/148; A61B 2017/320064; A61B 2018/00321; A61B 2018/00595; A61B 2018/0091; A61B 2018/1412

(57) ABSTRACT

A surgical device includes an elongated handle and at least one cutting assembly at an end of the handle. The at least one cutting assembly includes an elevator, a retainer located above the elevator, and at least one cutting blade positioned between the elevator and the retainer. The surgical device may be used to cut tissue, such as dura mater during brain surgery.

This PCT international application claims priority to U.S. Ser. No. 63/377,613, filed on 29 Sep. 2022 in the U.S. Patent and Trademark Office, the entirety of which is incorporated herein by reference.

### FIELD OF THE INVENTION

The present invention relates to a surgical device for cutting tissue. In particular, the surgical device may be used during brain surgery for cutting dura mater.

### BACKGROUND OF THE INVENTION

Typically, during brain surgery a dural opening is performed by two surgeons. One surgeon retracts the opened dura, while the other surgeon uses either scissors or a dental tool with a cutting surface. Both steps take time and risk damaging the brain below.

The surgical device of the present invention is useful to cut the dura mater during brain surgery (e.g., during trauma surgery, tumor removal surgery, and the like) where access to the subdural space is needed. The surgical device is particularly suitable to reduce risk of damaging brain matter below the dura level, while reducing the time needed to open the dura mater.

The surgical device of the present invention isolates and houses a cutting blade in a way that permits a safe and easy way to cut through the dura mater only, and only to the extent needed, by a single surgeon. No power source is needed, and the single surgeon has maximum control of the device, which provides an additional margin of safety.

### SUMMARY OF THE INVENTION

The invention provides in a first embodiment a surgical device comprising an elongated handle and at least one cutting assembly at an end of the handle. The at least one cutting assembly includes an elevator; a retainer positioned or located above the elevator; and at least one cutting blade between the elevator and the retainer and recessed from an opening formed by distal ends of the elevator and retainer.

The invention provides in a second embodiment further to any of the previous embodiments a surgical device wherein the elevator has a length that is greater than that of the retainer.

The invention provides in a third embodiment further to any of the previous embodiments a surgical device wherein a top side or edge and/or a bottom side or edge of the elevator has a curvature so that there is an upward curve at an outer or distal end.

The invention provides in a fourth embodiment further to any of the previous embodiments a surgical device wherein the retainer is angled with respect to a longitudinal axis formed by the handle.

The invention provides in a fifth embodiment further to any of the previous embodiments a surgical device wherein the at least one cutting blade is angled with respect to a longitudinal axis formed by the handle.

The invention provides in a sixth embodiment further to any of the previous embodiments a surgical device wherein at least one of the elevator or the retainer is substantially flat.

The invention provides in a seventh embodiment further to any of the previous embodiments a surgical device wherein the opening has a height of about 2 mm to about 5 mm.

The invention provides in an eighth embodiment further to any of the previous embodiments a surgical device wherein the handle has a length of about 10 cm to about 20 cm and/or the handle has a diameter of about 0.3 cm to about 1.0 cm.

The invention provides in a ninth embodiment further to any of the previous embodiments a surgical device wherein the retainer has a length of about 0.5 cm to about 1.5 cm and/or the elevator has a length of about 0.5 cm to about 2.0 cm.

The invention provides in a tenth embodiment further to any of the previous embodiments a surgical device further including means for attaching and/or exchanging the at least one cutting blade.

The invention provides in an eleventh embodiment further to any of the previous embodiment a surgical device wherein the elevator is configured to elevate dural tissue and the retainer is configured to retain dural tissue during brain surgery.

The invention provides in a twelfth embodiment further to any of the previous embodiments a surgical device having no power source and/or not being connectable to a power source.

The invention provides in a thirteenth embodiment further to any of the previous embodiments a surgical device having no cutting blade shield and/or no rotator mechanism.

The invention provides in a first method embodiment a method of cutting tissue including providing a surgical device of any of the previous embodiments; making an opening in tissue; and cutting the tissue with the at least one cutting blade.

The invention provides in a second method embodiment further to any of the previous method embodiments a method of cutting dura tissue including providing a surgical device of any of the previous embodiments; making an opening in dura tissue; lifting the dura tissue off at least one of brain, skin or fat with the dural elevator, while maintaining a lifted position of the dura tissue with the dural retainer; and cutting the dura tissue with the at least one cutting blade.

### BRIEF DESCRIPTION OF THE DRAWINGS

FIG. 1 shows a side view of a surgical device according to one embodiment of the present invention.

FIG. 2 shows a method of using the surgical device of one embodiment operated by the hand of a surgeon.

FIG. 3A shows a close-up side view of one end of the surgical device of FIG. 1.

FIG. 3B shows a front view of the surgical device of FIG. 3A.

FIG. 3C shows an elevated perspective view of a method of cutting dura mater using the surgical device of FIG. 3A.

FIG. 4 shows a perspective view of a surgical device according to another embodiment of the present invention.

### DETAILED DESCRIPTION OF THE INVENTION

The present invention is directed to a surgical device for cutting tissue. In particular, the surgical device may be used in brain surgery for cutting the dura mater. However, it is readily apparent that the surgical device may be used in other types of surgery for cutting any desired tissue plane. For example, the surgical device may be used for cutting the fascial layer when performing fasciotomies.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

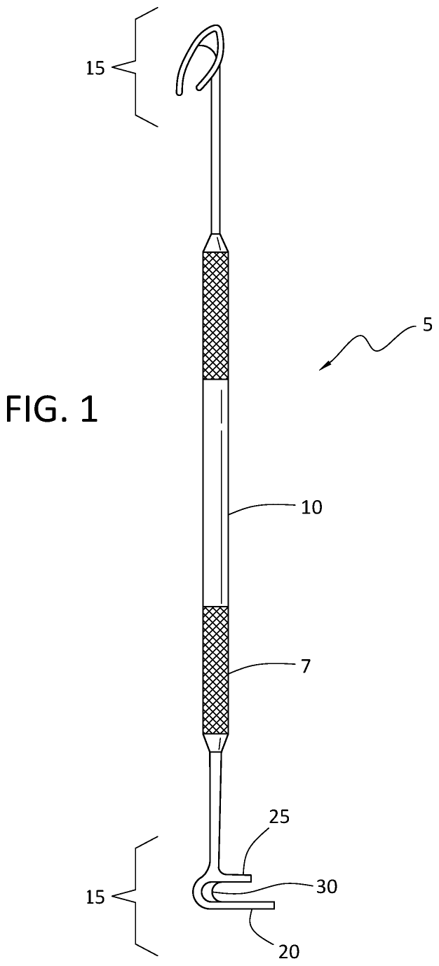

FIG. 1 shows a side view of surgical device 5 for cutting tissue according to one embodiment of the present invention. The surgical device 5 includes an elongated handle 10. In specific embodiments, the handle 10 may be about 10 cm to about 20 cm in length and have a diameter of about 0.3 cm to about 1.0 cm. The length and diameter may be selected to balance durability and ease of fine motor control.

In embodiments, the handle 10 may comprise a durable, non-bending material, such as metal (e.g., stainless steel, carbon steel) or a surgical-grade plastic (e.g., polycarbonate, polyethylene, polypropylene, acrylonitrile butadiene styrene). The body of the handle may include at least one knurled region 7, which provides a gripping surface to stabilize the instrument during surgery. The entire length of the surgical device may be about 15 cm to about 25 cm.

According to the present invention, the surgical device 5 has at least one cutting assembly 15. A cutting assembly may be located at one end, or both ends, of the handle 10. The at least one cutting assembly 15 includes an elevator 20, a retainer 25 positioned above the elevator 20, and at least one cutting blade 30 that extends between the elevator 20 and the retainer 25. In embodiments, the elevator and the retainer may not be laterally side-by-side.

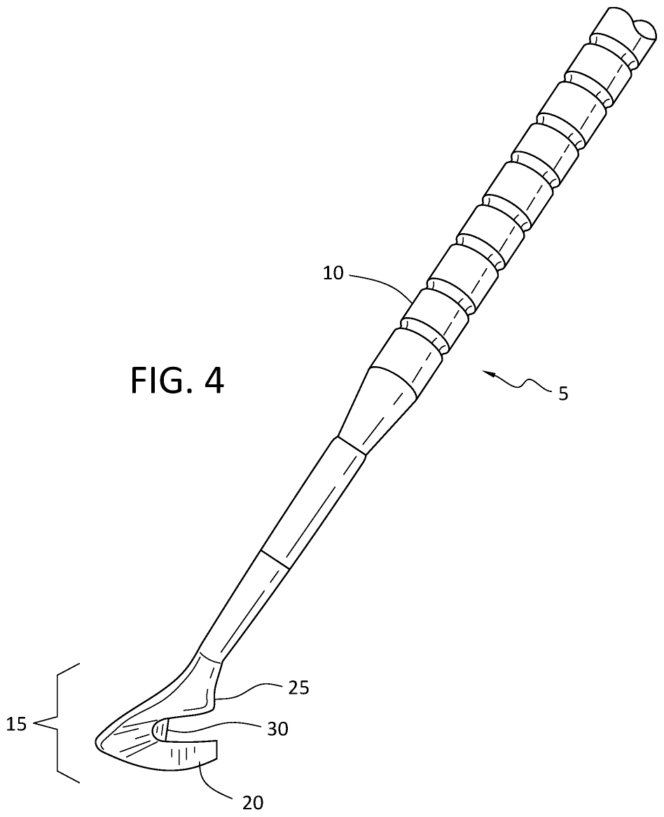

In a specific embodiment, and as shown in FIG. 1, the elevator 20 may be at substantially a right angle with respect to a longitudinal axis of the handle 10, and may be substantially flat. In another embodiment, as shown in FIG. 4, the elevator 20 may have a curvature on a bottom side or edge and/or on a top side or edge so that there is an upward curve at the outer or distal tip. The elevator 20 provides a surface for pulling up tissue, for example the dura, elevating it off the brain.

In embodiments, the elevator 20 may be about 0.5 cm to 2.0 cm in length, for example, 0.5 cm to 1.0 cm in length, thereby being long enough to elevate enough of the dura to visualize a surface to be cut, but not so long that a bone edge obstructs the movement of the device as the surgeon nears margins of the opening of the skull (craniotomy).

In specific embodiments, the retainer 25 may be about 0.5 cm to 1.5 cm in length. In embodiments, as shown in FIG. 1, the retainer 25 may extend outwardly from the handle 10 at substantially a right angle with respect to a longitudinal axis of the handle and at least partially over the elevator 20. The retainer 25 directs the dura or other similar tissue plane towards the cutting blade 30 as the surgeon guides the surgical device along a desired path to open the dura or cut tissue. In embodiments, the retainer 25 may be substantially flat. In another embodiment, as shown in FIG. 4, the retainer 25 or an edge thereof may be angled with respect to a longitudinal axis of the handle.

The at least one cutting blade 30 is positioned or located at a junction between the elevator 20 and retainer 30, recessed from an opening formed by distal ends of the elevator and retainer. The opening may be about 2 mm to about 5 mm, for example, about 2.5 mm to about 3 mm, in order to accommodate the thickness of the dura or other tissue, while also allowing the retainer 25 to protect the surrounding tissues from the at least one cutting blade 30. The cutting blade may be substantially perpendicular to the elevator and retainer (FIG. 1) or may be at an angle, if the elevator 20 is curved and/or if the retainer 25 is at an angle as discussed above (FIG. 4). In the present invention, neither the elevator nor the retainer acts as a cutting surface.

The at least one cutting blade 30 may be made of plastic or metal. In a specific embodiment, the at least one cutting blade 30 may be a standard #5 surgical blade. In embodiments, the entire cutting assembly 15 may be positioned at a right angle, or may be angled upwards or downwards (e.g., from 20° to 50°), with respect to a longitudinal axis of the handle 10 in order to provide a surgeon flexibility while cutting tissue from different angles or different positions. In embodiments, the cutting assembly 15 may be movable or adjustable to whatever angle the surgeon desires.

In embodiments, the elevator and/or retainer do not have a portion or protrusion extending upwardly or downwardly from an edge or surface therefrom into the opening which is configured to protect tissue from the at least one cutting blade. Thus, in embodiments, there is no cutting blade shield. There is also no rotator mechanism for deploying or retracting the cutting blade or any shield.

In a specific embodiment the at least one cutting blade 30 may be an electrocautery blade (e.g., a unipolar or monopolar cautery blade). This configuration allows for cutting a vascular surface while simultaneously cauterizing any bleeding. An electrocautery blade would require a power source.

In specific embodiments, the handle 10 may include a means for attaching and/or exchanging the at least one cutting blade 30 including, but not limited to, one or more screws, bolts, or grooves. The at least one cutting blade 30 may need to be replaced when it becomes dull or when the surgical device must be sterilized for a new surgery and/or for a new patient.

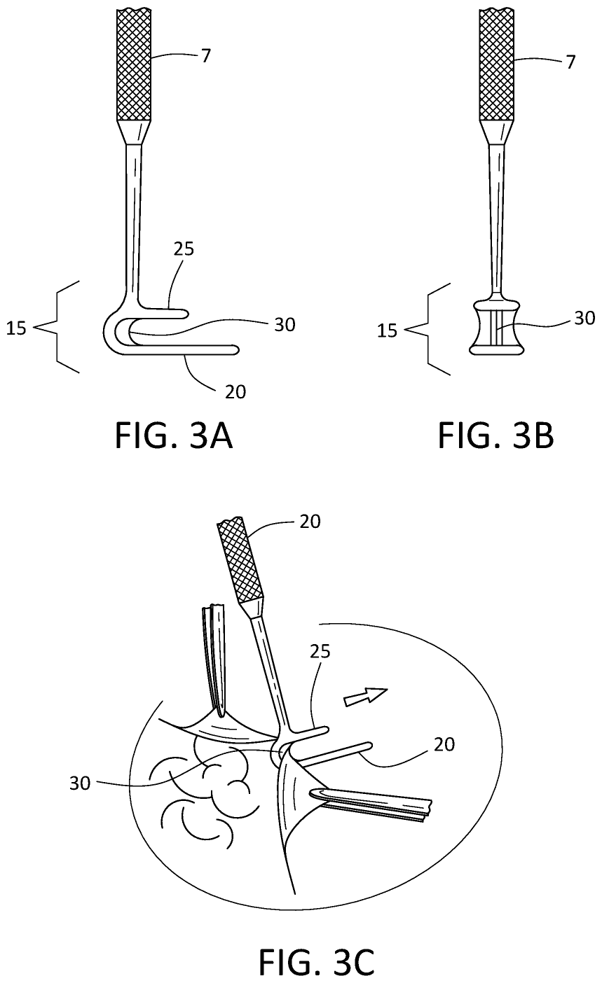

FIG. 3A shows a close-of view of the cutting assembly of the surgical device 5 of one embodiment of the present invention. FIG. 3B shows a front view of the surgical device 5 of FIG. 3A.

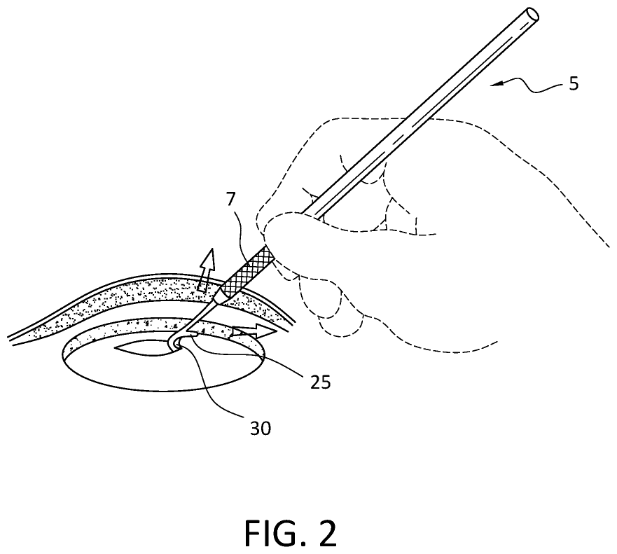

FIGS. 2 and 3C schematically show methods of using the surgical device of the present invention as operated by the hand of a surgeon. A view of the brain includes skin and fat and skull layers, beneath which are the dura mater and the brain. As shown, a surgeon pulls the surgical device 5 while maintaining gentle upward pressure. Upward pressure is maintained to elevate the dura off the surface of the brain using the elevator 20, while maintaining a position via the retainer 25. The at least one cutting blade 30 is used to cut the dura, without harming other surrounding tissue or structures (e.g., the brain structures directly underneath the dura layer).

### INDUSTRIAL APPLICABILITY

The present invention is a surgical device for cutting tissue, which in particular, may be used in brain surgery for cutting the dura mater.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

1. A surgical device, comprising:
an elongated handle; and
a cutting assembly at an end of the handle;
wherein the at least one cutting assembly comprises:
an elevator, wherein a top side or edge and/or a bottom side or edge of the elevator has an upward curvature extending along a length of the elevator to an outer or distal end,
a retainer positioned or located above the elevator, wherein the retainer is angled with respect to a longitudinal axis formed by the handle, and
at least one cutting blade between the elevator and the retainer and recessed from an opening formed by distal ends of the elevator and retainer.

2. The surgical device of claim 1, wherein the elevator has a length that is greater than that of the retainer.

3-4. (canceled)

5. The surgical device of claim 1, wherein the at least one cutting blade is angled.

6. (canceled)

7. The surgical device of claim 1 wherein the opening has a height of about 2 mm to about 5 mm.

8. The surgical device of claim 1, wherein the handle has a length of about 10 cm to about 20 cm.

9. The surgical device of claim 1, wherein the handle has a diameter of about 0.3 cm to about 1.0 cm.

10. The surgical device of claim 1, wherein the retainer has a length of about 0.5 cm to about 1.5 cm.

11. The surgical device of claim 1, wherein the elevator has a length of about 0.5 cm to about 2.0 cm.

12. The surgical device of claim 1, further comprising means for attaching and/or exchanging the at least one cutting blade.

13. The surgical device of claim 1, wherein the at least one cutting blade comprises an electrocautery blade.

14. The surgical device of claim 1, wherein the elevator is configured to elevate dural tissue and the retainer is configured to retain dural tissue during brain surgery.

15. The surgical device of claim 1, wherein the device has no power source and/or is not connectable to a power source.

16. The surgical device of claim 1, wherein there is no cutting blade shield and/or no rotator mechanism.

17. A method of cutting tissue, comprising:
providing the surgical device of claim 1;
making an opening in tissue; and
cutting the tissue with the at least one cutting blade.

18. The method of cutting tissue according to claim 17, wherein the tissue comprises dura tissue and said method further comprises lifting the dura tissue off the brain with the elevator, while maintaining a lifted position of the dura tissue with the retainer.

19. The method of claim 17, wherein the method is performed by a single surgeon.

20. The method of claim 17, wherein the method is performed without a power source for the surgical device.

21. The surgical device of claim 1, wherein the cutting assembly is angled upwards from 20° to 50° with respect to a longitudinal axis of the handle.

22. The surgical device of claim 1, wherein the cutting assembly is movable or adjustable with respect to a longitudinal axis of the handle.
